(19) United States
(12) Patent Application Publication
(10) Pub. No.: US 2022/0039650 A1
(43) Pub. Date: Feb. 10, 2022

(54) FUNDUS IMAGING APPARATUS

(71) Applicant: NIDEK CO., LTD., Gamagori (JP)

(72) Inventors: Shinya IWATA, Gamagori (JP); Junpei NISHIYAMA, Gamagori (JP); Norimasa SATAKE, Gamagori (JP)

(73) Assignee: NIDEK CO., LTD., Gamagori (JP)

(21) Appl. No.: 17/391,155

(22) Filed: Aug. 2, 2021

(30) Foreign Application Priority Data
Aug. 4, 2020 (JP) 2020-132378
Sep. 29, 2020 (JP) 2020-164190

(51) Int. Cl. A61B 3/12; A61B 3/10; A61B 3/00

(52) U.S. Cl. CPC A61B 3/12; A61B 3/0008; A61B 3/102

(57) ABSTRACT

A fundus imaging apparatus includes an OCT optical system for acquiring OCT data based on measurement light irradiated on a fundus of a subject eye and reference light, and a front imaging optical system for capturing a front image of the fundus of the subject eye by scanning light on the fundus. The scanning light scanned on the fundus of the subject eye in the front imaging optical system has a wavelength, which is longer than λ=850 nm, in a wavelength region in which a scanning light is unlikely to be visible for the subject eye.

### CROSS-REFERENCE TO RELATED APPLICATION

This application claims priority from Japanese Patent Applications No. 2020-132378 filed on Aug. 4, 2020 and No. 2020-164190 filed on Sep. 29, 2020, the entire subject-matter of which is incorporated herein by reference.

### TECHNICAL FIELD

The present disclosure relates to a fundus imaging apparatus that images a fundus of a subject eye.

### BACKGROUND

As the fundus imaging apparatus, not only an OCT device for capturing a tomographic image of a fundus of a subject eye but also a device for capturing a front image of the fundus of the subject eye have been known. A fundus camera is generally used as the fundus imaging apparatus for capturing a front image of the fundus of the subject eye. In recent years, a fundus imaging apparatus for scanning a light on the fundus is also used in clinical practice. The scanning fundus imaging apparatus may be used as a single device that scans visible light to capture a color front image of the fundus, or may be used as a fundus observation system such as an OCT or a perimeter (refer to, for example, JP-A-2016-104105).

In the scanning fundus imaging apparatus, light having a wavelength characteristic relatively close to that of visible light (for example, λ=780 nm) is used for fundus observation.

Meanwhile, the human eye has the visual sensitivity in 850 nm or less wavelength. Therefore, in the OCT device, SS-OCT having a central wavelength between λ=1000 nm to 1100 nm inclusive has been known in order to avoid tracking of the subject eye when acquiring OCT data.

However, even in the above SS-OCT, as for the light for observing the fundus, the light having a wavelength characteristic relatively close to that of visible light is currently used. The present inventors have found that visual fixation of the subject eye is not stable because the subject eye tracks the scanning light from the fundus observation light so that the instable visual fixation adversely affects the acquisition of OCT data or the acquisition of a color front image of the fundus.

### SUMMARY

An object of the present disclosure is to provide a fundus imaging apparatus that enables to stabilize visual fixation of a subject eye to suitably image a fundus of the subject eye.

According to a first aspect of the present disclosure, there is provided a fundus imaging apparatus including: an OCT optical system configured to be used for acquiring OCT data based on measurement light irradiated on a fundus of a subject eye and reference light; and a front imaging optical system configured to be used for capturing a front image of the fundus of the subject eye by scanning light on the fundus, in which the scanning light scanned on the fundus of the subject eye in the front imaging optical system has a wavelength, which is longer than λ=850 nm, in a wavelength region in which a scanning light is unlikely to be visible for the subject eye.

According to a second aspect of the present disclosure, there is provided a fundus imaging apparatus including: a measurement optical system that projects a stimulus point onto a fundus of a subject eye with visible light to measure a visual field; and a front imaging optical system configured to be used for capturing a front image of the fundus of the subject eye by scanning light on the fundus, in which the scanning light scanned on the fundus of the subject eye in the front imaging optical system has a wavelength, which is longer than λ=850 nm, in a wavelength region in which a scanning light is unlikely to be visible for the subject eye.

According to a third aspect of the present disclosure, there is provided a fundus imaging apparatus including: a visible imaging optical system configured to be used for imaging a fundus of a subject eye with visible light; and a front imaging optical system configured to be used for capturing a front image of the fundus of the subject eye by scanning light on the fundus, in which the scanning light scanned on the fundus of the subject eye in the front imaging optical system has a wavelength, which is longer than λ=850 nm, in a wavelength region in which a scanning light is unlikely to be visible for the subject eye.

According to the present disclosure, it is possible to stabilize the visual fixation of the subject eye and suitably image the fundus.

### BRIEF DESCRIPTION OF DRAWINGS

FIG. 1 is a diagram showing an optical system of a fundus imaging apparatus according to a first example of a first embodiment.

FIG. 2 is a diagram showing an optical system of a fundus imaging apparatus according to a second example of the first embodiment.

FIG. 3 is a diagram showing an example of a cut-on wavelength of a second wavelength separation member according to the first example.

FIG. 4 is a diagram showing an example of a cut-on wavelength of a first wavelength separation member according to the first example.

FIG. 5 is a diagram showing an optical system of a fundus imaging apparatus according to a third example.

FIG. 6 is a diagram showing an optical system of a fundus imaging apparatus according to a fourth example.

FIG. 7 is a block diagram showing an example of a control system according to an example of the second embodiment.

FIG. 8 is a diagram showing an example of a display screen of an apparatus according to the example of the second embodiment.

FIG. 9 is a flowchart showing an example of detecting a change in OCT data before and after light stimulation.

### DETAILED DESCRIPTION

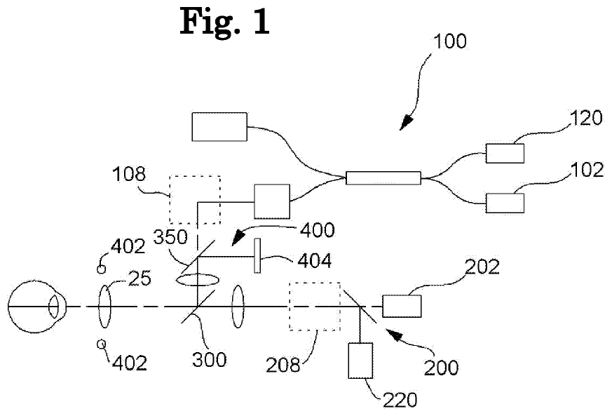

Examples of the embodiments of the present disclosure will be described with reference to the drawings. FIGS. 1 to 9 are diagrams according to examples of the present embodiment. The items classified by < >in the following description can be used independently or in relation to one another.

Hereinafter, a fundus imaging apparatus according to the first embodiment will be described. The fundus imaging apparatus may include an OCT optical system and a front imaging optical system.

<OCT Optical System>

The OCT optical system may be, for example, an OCT optical system for acquiring OCT data by the measurement light irradiated on a fundus of a subject eye and the reference light. Further, the OCT optical system may include an optical scanner which scans the measurement light on the fundus of the subject eye.

<Front Imaging Optical System>

The front imaging optical system may be, for example, a front imaging optical system for capturing a front image of the fundus by scanning light on the fundus of the subject eye. Further, the front imaging optical system may include an optical scanner which scans light on the fundus.

The front imaging optical system may have, for example, a configuration in which the spot light is two-dimensionally scanned on the fundus, or a configuration in which the slit light is one-dimensionally scanned. Further, the optical scanner may be, for example, a scanning mirror (for example, a galvano mirror or a resonant scanner), or may have a configuration in which a chopper having a slit is rotationally driven.

The front imaging optical system may be, for example, an SLO optical system based on a scanning laser ophthalmoscope (SLO), and a point sensor or a line sensor may be used as a light receiving element. The front imaging optical system may be a slit scan type fundus camera, or a two-dimensional imaging element may be used as a light receiving element.

The scanning light scanned on the fundus in the front imaging optical system may have a wavelength, which is longer than λ=850 nm, in a wavelength region in which the scanning light is unlikely to be visible for the subject eye. Thereby, the subject eye is unlikely to track the light scanning of the front imaging optical system 200, for example. As a result, it is possible to acquire OCT data in a state where visual fixation of the subject eye is stable. Therefore, favorable OCT data can be acquired.

More preferably, the scanning light scanned on the fundus of the subject eye in the front imaging optical system may have a wavelength, which is longer than λ=900 nm, in an invisible region for the subject eye. Thereby, for example, it is possible to prevent the subject eye from tracking the light scanning of the front imaging optical system. Therefore, favorable OCT data can be acquired. As a result, it is possible to acquire OCT data in a state in which the visual fixation of the subject eye is more stable. Therefore, more favorable OCT data can be acquired.

For example, the scanning light may be irradiated on the fundus concurrently during the acquisition of the OCT data acquired by the OCT optical system. Thereby, for example, during the acquisition of the OCT data, it is possible to avoid the tracking of the subject eye by the light scanning of the front imaging optical system. Therefore, favorable OCT data can be acquired. Further, the scanning light may be irradiated on the fundus at a timing other than the acquisition of the OCT data acquired by the OCT optical system. Thereby, for example, it is possible to avoid the tracking of the subject eye in the front imaging optical system, and it is possible to start the acquisition of the OCT data in a state where the visual fixation of the subject eye is stable.

In the above, in a case where the scanning light is in a wavelength region in which the scanning light is unlikely to be visible (preferably, an invisible region), a light source that emits light having a central wavelength may be used as a light source used in the front imaging optical system. The central wavelength is in a wavelength band longer than λ=850 nm (preferably λ=900 nm). Thereby, when the front image is obtained by the scanning light, the central wavelength having high wavelength efficiency can be used. Therefore, efficient imaging is possible while reducing the possibility that the subject eye tracks the scanning light. In this case, the lower limit of the wavelength band of the light emitted from the light source may be a wavelength longer than 850 nm, or light having a wavelength shorter than 850 nm may be cut in the optical path. Thereby, it is also possible to reduce the possibility that the scanning light will be tracked by light other than the central wavelength. The wavelength band of the light source of the front imaging optical system can be defined based on, for example, the full width at half maximum.

The configuration in which the scanning light is unlikely to be visible (preferably in an invisible region) is not limited to the above configuration. For example, a light source emitting light, of which the central wavelength is shorter than λ=850 nm (preferably λ=900 nm) and which has a wavelength region longer than λ=850 nm as the upper limit of the emission wavelength, is used, and light having a wavelength shorter than λ=850 nm is cut in the optical path by a cut filter or the like. Thereby, light having a wavelength longer than λ=850 nm may be scanned on the fundus.

In the fundus imaging apparatus including the OCT optical system and the front imaging optical system as described above, the light source used for the OCT optical system may be a light source that emits the measurement light having a central wavelength between λ=1000 nm to 1100 nm inclusive. Thereby, for example, in addition to the scanning light on the fundus having the above wavelength characteristics, the measurement light irradiated on the fundus is in the invisible region. Therefore, it is possible to more appropriately avoid the tracking of the subject eye when obtaining the OCT data.

Hereinafter, a fundus imaging apparatus according to the second embodiment will be described.

As the fundus imaging apparatus, there is a known apparatus which includes a visual field measurement optical system and an OCT optical system and which is capable of performing a visual field examination of the fundus and the OCT imaging of the fundus in one device (for example, refer to JP-A-2012-100713 and JP-A-2012-100714).

By the way, it takes a long time to perform the visual field examination using a perimeter among ophthalmic examinations, and the burden on the subject or the examiner is large. On the other hand, even in tomography using OCT, the time for examination, such as tomography under various imaging conditions or OCT angiography, may be long. As a result, a series of fundus examinations consisting of visual field examination and OCT imaging requires a long examination time. However, in the case of the fundus imaging apparatus described in JP-A-2012-100713 and JP-A-2012-100714, the visual field examination and the OCT imaging are separated in time. Thus, still there is an issue about the long examination time.

It is desirable for the fundus imaging apparatus according to the second embodiment to more efficiently perform a visual field examination and an OCT examination on the fundus.

The fundus imaging apparatus according to the second embodiment may include a visual field measurement optical system and an OCT optical system. Further, the fundus imaging apparatus may include a controller that controls the visual field measurement optical system and the OCT optical system. No particular description will be given to the parts common to the first embodiment.

<Visual Field Measurement Optical System>

The visual field measurement optical system (hereinafter referred to as measurement optical system) may be, for example, a measurement optical system for projecting a stimulus point onto the fundus of the subject eye with visible light to perform visual field measurement, and may include a visible light source for stimulating the fundus of the subject eye.

The visual field measurement optical system may include a visual target projection optical system that projects a visual examination target (visual stimulation target) for visual field measurement. In this case, the visual examination target may be projected as a stimulus point of the fundus through the display control of the display, and the visual examination target may be projected as the stimulus point of the fundus by controlling the light from the visible light source by the optical scanner. It is apparent that the present invention is not limited to this. The visual examination target may be projected as a stimulus point of the fundus by arranging a plurality of visible light sources two-dimensionally and controlling the lighting position of the visible light sources. In this case, the visual target projection optical system may project a visual fixation target as the visual fixation target of the subject eye during visual field measurement.

<OCT Optical System>

The light source used in the OCT optical system is, for example, a light source that emits measurement light having a central wavelength in a wavelength region which is an invisible region for the subject eye (for example, a light source which emits measurement light having a central wavelength between λ=1000 nm to 1100 nm inclusive). Thereby, for example, even in a case where the OCT data is collected during the visual field examination, it is possible to prevent the subject eye from tracking the measurement light of the OCT optical system. Therefore, a favorable visual field examination result can be obtained.

<Acquisition of OCT Data During Visual Field Examination>

For example, the controller may control the measurement optical system based on a visual field examination pattern in which a plurality of stimulus points are set in advance. Further, the controller may collect the OCT data of the fundus of the subject eye by controlling the OCT optical system while each stimulus point is projected onto the fundus of the subject eye.

Thereby, for example, the OCT data is collected during the visual field examination. Therefore, it is possible to complete a series of fundus examinations including the visual field examination and OCT imaging in a short time. As a result, the burden on the examiner or the subject is reduced. On the other side, the relatively time-consuming OCT imaging is performed during the visual field examination to reduce the burden on the examiner or the subject.

The collected OCT data may be, for example, various OCT data which are OCT data acquired under various imaging conditions. The various OCT data can be obtained reliably by capturing OCT images during a visual field examination regardless of an imaging time. In this case, various OCT data include, for example, at least one of a plurality of OCT data pieces having different scanning patterns, a plurality of OCT data pieces having different scanning lines (for example, three-dimensional OCT data obtained by raster scanning), and a plurality of OCT data pieces having different imaging modes. Further, the collected OCT data may be, for example, OCT data acquired under a single imaging condition. The OCT data that requires imaging time even under a single imaging condition by OCT imaging during a visual field examination can be acquired reliably regardless of the imaging time. In this case, the OCT data that requires the imaging time includes at least one of a plurality of OCT data pieces at the same position (for example, data that is the basis of the averaging image), a plurality of OCT data pieces that differ in time at the same position (for example, the data on which OCT motion contrast data (OCT angio data) is based, the OCT Doppler data, and the like), and the wide-range and high-density OCT data.

In a case where collecting the OCT data, for example, the controller may collect the OCT data in accordance with a preset imaging control pattern before the start of visual field measurement, and may collect the OCT data by setting an imaging control pattern arbitrarily during visual field measurement. It is apparent that these collection methods may be combined. For example, collection by a preset imaging control pattern and collection by an imaging pattern set during visual field measurement may be performed separately before and after. In this case, the imaging control pattern may be an imaging control pattern composed of a single or a plurality of imaging conditions.

<Setting of OCT Acquisition Position Using Visual Field Measurement Results>

In a case where the imaging control pattern is arbitrarily set during the visual field measurement, the controller may be able to set the acquisition position of the OCT data by the OCT optical system, based on the visual field measurement result measured while each stimulus point is projected onto the fundus of the subject eye. Thereby, for example, by setting the acquisition position of the OCT data in accordance with the visual field measurement result, the OCT data acquisition during the visual field examination can be performed more effectively.

In this case, the controller may also automatically sets the acquisition position of the OCT data in accordance with the visual field measurement (for example, the controller detects the abnormal part on the fundus in the visual field measurement, and sets the abnormal part as an acquisition position of the OCT data). Further, the controller is not limited to this setting. The controller may set the acquisition position of the OCT data, based on the visual field measurement result, by displaying the visual field measurement result on the display unit and receiving an operation instruction from the examiner to set the acquisition position of the OCT data. In this case, as the visual field measurement result, the visual field measurement result may be directly used, or the analysis result obtained by analyzing the visual field measurement result may be used.

<Setting of Visual Field Measurement Position Using OCT Data>

The controller may be able to set the position of the stimulus point projected by the measurement optical system, based on the OCT data collected while each stimulus point is projected onto the fundus of the subject eye. Thereby, for example, the position of the visual field measurement is set based on the collection result of the OCT data obtained during the visual field examination. Therefore, the examination time of the visual field examination itself can be shortened.

In this case, the controller may automatically set the position of the stimulus point in accordance with the collection result of the OCT data (for example, the controller detects an abnormal part on the fundus in the OCT data, and sets the abnormal part as the position of the stimulus point). Further, the controller is not limited to this. The controller may set the position of the stimulus point, based on the OCT data, by displaying the collection result of the OCT data on the display unit and receiving an operation instruction from the examiner to set the position of the stimulus point. In this case, as the collection result of the OCT data, various OCT data may be directly used, or the analysis result obtained by analyzing various OCT data may be used.

The setting of the position of the stimulus point is not limited. The controller may change the luminance of the stimulus point by the measurement optical system stepwise based on the collection result of the OCT data. For example, the controller may change stepwise the luminance in accordance with the degree of the disease in the abnormal part. The controller may decrease the luminance in a case where the disease is severe, and may increase the luminance in a case where the disease is not severe. The relationship between the degree of disease and the luminance can be obtained in advance by experiments and simulations. For example, the luminance may be changed stepwise in accordance with the thickness of the fundus. For example, in a case where the specific retinal layer is thin, the luminance may be lowered, and in a case where the specific retinal layer is thick, the luminance may be increased.

<OCT Measurement Before and After Light Stimulation During Visual Field Measurement>

The controller may perform light stimulation on the fundus of the subject eye by controlling the measurement optical system or a light stimulation unit different from the measurement optical system, and may detect a change in the OCT data before and after the light stimulation by controlling the OCT optical system. Thereby, for example, the change in OCT data before and after light stimulation during visual field measurement can be obtained. Therefore, in addition to the visual field measurement results, structural or functional change in the retina before and after stimulation can be confirmed in more detail.

In a case where a light stimulation unit different from the measurement optical system is used, for example, an external visual fixation lamp, a visible light source for fundus imaging, or the like may be used. In this case, the controller may acquire the OCT data by the OCT optical system in accordance with the light emission timing of the light stimulation unit.

For example, the controller may acquire the OCT data before and after light stimulation and detect a temporal change in the obtained OCT data. In addition to before and after light stimulation, the controller may acquire the OCT data during light stimulation.

<Acquisition of OCT Data Including Visual Field Measurement Position>

The controller may change the acquisition position of the OCT data in accordance with the position of the stimulus point so that the OCT data at each stimulus point is acquired. Thereby, for example, the OCT data corresponding to each stimulus point can be smoothly acquired.

In this case, for example, the controller may change the acquisition position of the OCT data in accordance with the position of the stimulus point, may acquire the OCT data before and after the light stimulation by each stimulus point, and may detect the change in the obtained OCT data. Thereby, for example, the change of the OCT data before and after the light stimulation corresponding to each stimulus point can be smoothly acquired.

### EXAMPLE

Hereinafter, examples according to the first embodiment will be described with reference to the drawings.

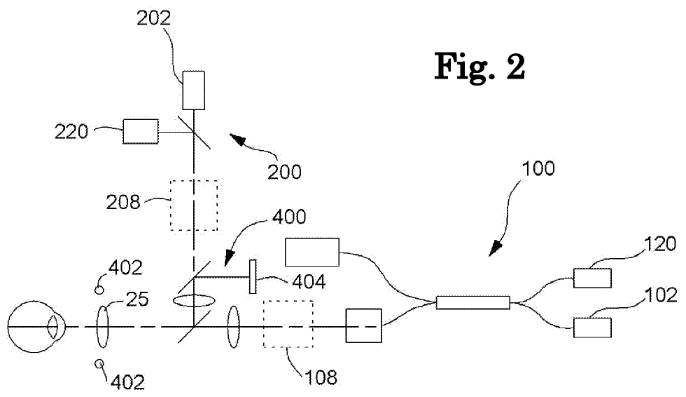

FIGS. 1 and 2 are diagrams showing optical systems according to the present example, respectively. A fundus imaging apparatus according to the present example mainly includes an objective lens 25, an OCT optical system 100, and a front imaging optical system 200. The fundus imaging apparatus may further include a visual fixation optical system that presents a visual fixation target for performing visual fixation of the subject eye. The visual fixation optical system may be provided as a part of the front imaging optical system 200, or may be provided separately.

The objective lens 25 may be disposed in front of the subject eye, for example, or may be composed of one or a plurality of lenses. In this case, the objective lens 25 may be shared between the OCT optical system 100 and the front imaging optical system 200.

<OCT Optical System>

The OCT optical system 100 may be provided to acquire OCT data of the fundus of the subject eye through, for example, the objective lens 25 by using a technique of optical interference.

More specifically, the OCT optical system 100 mainly includes a light source 102, a detector 120, and an optical scanner 108. The light emitted from the light source 102 is divided into measurement light and reference light by a splitter. The measurement light is guided to the fundus Ef through the measurement optical path, and then is guided to the detector 120 through the measurement optical path. The reference light is guided to the detector 120 through the reference optical path. The detector 120 detects an interference state between the reference light and the measurement light irradiated on the fundus of the subject eye.

For example, the OCT optical system may be a Fourier domain OCT such as Spectral-domain OCT (SD-OCT) or Swept-source OCT (SS-OCT), or may be a Time-domain OCT (TD-OCT).

The optical scanner 108 is disposed in the measurement optical path to scan the measurement light on the fundus of the subject eye. The optical scanner 108 may repeatedly scan the measurement light on the subject eye.

The fundus imaging apparatus is able to obtain OCT data, based on the detection signal from the detector 120 at each scanning position of the optical scanner 108.

In the present example, as the light source 102 (measurement light source) of the OCT optical system 100, a light source that emits light having a central wavelength between λ=1000 nm to 1100 nm inclusive may be used. In this case, for example, the light source 102 may be a wavelength sweep light source (SS-OCT light source). A light source having a wavelength band of ±30 to 60 nm with respect to the central wavelength as the bandwidth with respect to the central wavelength may be used. For example, the lower limit of the wavelength band of the light emitted from the light source 102 may be shorter than λ=1000 nm.

In this case, the light scanned on the fundus by the OCT optical system 100 makes the subject eye unlikely to track the light scanning of the OCT optical system 100, which is an invisible region for the subject eye. Therefore, it is possible to acquire OCT data in a state in which the visual fixation of the subject eye is stable. As a result, favorable OCT data can be acquired.

<Front Imaging Optical System>

The front imaging optical system 200 may be provided, for example, to obtain a front image of the fundus of the subject eye through the objective lens 25. In this case, the front imaging optical system 200 may be a scanning type imaging optical system (for example, an SLO optical system) that captures a front image of the fundus by scanning light on the fundus of the subject eye.

The front imaging optical system 200 mainly includes a light source (for example, a laser light source) 202, a light receiving element 220, and an optical scanner 208. In this case, the front imaging optical system 200 projects the light from the light source 202 to the fundus, and receives the fundus reflected light by the light from the light source 202 through the light receiving element 220.

The optical scanner 208 is disposed in the optical path of the front imaging optical system 200 (for example, the pupil conjugate position), and is used to two-dimensionally scan the light from the light source 202 on the fundus. The optical scanner 208 is formed of, for example, a combination of two optical scanners. One optical scanner performs light scanning in the main scanning direction, and the other optical scanner performs light scanning in the sub-scanning direction.

The fundus imaging apparatus is able to obtain a front image of the fundus, based on the detection signal from the light receiving element 220 at each scanning position of the optical scanner 208.

The front imaging optical system 200 may be used, for example, as a fundus observation optical system for observing the fundus of the subject eye from the front direction. An examiner is able to be set to acquire the position of the OCT data on the fundus while viewing the front image of the fundus. Further, by detecting the positional deviation of the fundus based on the front image of the fundus and controlling the OCT optical system 100 based on the detection result, the position of the OCT data on the fundus may be corrected (the so-called tracking).

In the present example, as the light source 202 of the front imaging optical system 200, a light source which emits light having a central wavelength in a wavelength band longer than λ=850 nm and shorter than the measurement light of the OCT optical system 100 (for example, λ=990 nm to 1100 nm) may be used. A light source, which has a wavelength band of ±10 nm with respect to the central wavelength as the bandwidth with respect to the central wavelength so as to have laser characteristics, may be used. It is apparent that a wider band light source may be used.

In this case, the scanning light scanned on the fundus in the front imaging optical system 200 has a wavelength, which is longer than λ=850 nm, in a wavelength region in which the scanning light is unlikely to be visible for the subject eye. Therefore, the subject eye is unlikely to track the light scanning of the front imaging optical system 200. Therefore, it is possible to acquire the OCT data in a state where the visual fixation of the subject eye is stable. As a result, favorable OCT data can be acquired.

<First Wavelength Separation Member as Wavelength Selective Splitter>

It should be noted that a first wavelength separation member 300 (for example, dichroic mirror) for performing wavelength separation in order to divide the optical path of the measurement light in the OCT optical system 100 and the optical path of the scanning light in the front imaging optical system 200 may be provided as a wavelength selective splitter. For example, the first wavelength separation member 300 may reflect the measurement light used in the OCT optical system 100 and transmit the scanning light used in the front imaging optical system 200 (refer to FIG. 1). Further, the present invention is not limited to this, and the first wavelength separation member 300 may transmit the measurement light used in the OCT optical system 100 and reflect the scanning light used in the front imaging optical system 200 (refer to FIG. 2).

In the present example, the first wavelength separation member 300 may have, for example, a wavelength selection characteristic for dividing the measurement light of the OCT optical system 100 and the scanning light scanned in the front imaging optical system 200 and having a wavelength band longer than λ=850 nm. A cut-on wavelength is set in the first wavelength separation member 300 to be in a wavelength region in which a scanning light is unlikely to be visible for the subject eye, which is longer in wavelength than the wavelength band of the scanning light in the front imaging optical system 200, and which is shorter in wavelength than the wavelength band of the measurement light in the OCT optical system 100.

In the above configuration, more preferably, as the light source 202 of the front imaging optical system 200, a light source may be used which emits light having a central wavelength in a wavelength band longer than λ=900 nm and shorter than the measurement light (for example, λ=990 nm to 1100 nm inclusive) of the OCT optical system 100.

In this case, the scanning light scanned on the fundus in the front imaging optical system 200 has a longer wavelength than λ=900 nm, which is in an invisible region for the subject eye. Therefore, it is possible to prevent the subject eye from tracking the light scanning of the front imaging optical system 200. Therefore, in a case where the OCT data is acquired in parallel with the observation of the fundus by the front imaging optical system 200 (for example, tracking control), or in a case where the measurement light using the front imaging optical system 200 is positioned in a step before the acquisition of the OCT data or the imaging timing is determined, the visual fixation of the subject eye is more stable. As a result, more favorable OCT data can be acquired. As described above, it is possible to further stabilize the visual fixation when obtaining the OCT data by using the light in the invisible region including the scanning light scanned on the fundus by the OCT optical system 100.

In this case, the first wavelength separation member 300 may have, for example, a wavelength selection characteristic for dividing into the measurement light of the OCT optical system 100 and the scanning light scanned in the front imaging optical system 200 and having a wavelength band, which is longer than λ=900 nm. A cut-on wavelength is set in the first wavelength separation member 300 to be in the invisible region for the subject eye, which is longer in wavelength than the wavelength band of the scanning light in the front imaging optical system 200, and which is shorter in wavelength than the wavelength band of the measurement light in the OCT optical system 100.

<Anterior Segment Observation Optical System>

In addition, in the fundus imaging apparatus according to the present example, for example, an anterior segment observation optical system 400 for observing the anterior segment of the subject eye may be provided. In this case, the anterior segment observation optical system 400 may mainly include an anterior segment illumination light source 402 and an imaging element 404. The anterior segment observation optical system 400 guides the reflected light of the anterior segment of the subject eye illuminated by the anterior segment illumination light source 402 to the imaging element 404 (for example, a two-dimensional imaging element) through the objective lens 25. The imaging element 404 may be disposed at a position conjugate to the anterior segment of the eye, for example.

The fundus imaging apparatus is able to obtain a front image of the anterior segment of the eye based on the imaging signal from the imaging element 404. As the anterior segment illumination light source 402, for example, a light source having a central wavelength in a wavelength band of λ=930 nm to 950 nm inclusive may be used. Further, the present invention is not limited to this, as the anterior segment illumination light source 402, a light source, which has a central wavelength in a wavelength band deviating from λ=930 nm to 950 nm inclusive and emits light including at least a wavelength band of λ=930 nm to 950 nm inclusive, may be used. In the present example, the anterior segment observation optical system 400 guides reflected light including at least a wavelength band of λ=930 nm to 950 nm inclusive to the imaging element 404.

<First Wavelength Separation Member as Wavelength Selective Splitter>

In the fundus imaging apparatus, in a case where the anterior segment observation optical system 400 is provided, for example, a first wavelength separation member 300 performs wavelength separation to divide an optical path of the measurement light in the OCT optical system 100 and the optical path of the scanning light on the front imaging optical system 200 and to divide an optical path of the anterior segment observation optical system 400 with respect to one of optical paths of the front imaging optical system 200 and the OCT optical system 100.

The first wavelength separation member 300 may reflect the measurement light used in the OCT optical system 100 and the anterior segment reflected light using the anterior segment illumination light source 402 and may transmit the scanning light used in the front imaging optical system 200 (refer to FIG. 1). In this case, the first wavelength separation member 300 may have, for example, a wavelength selection characteristic for dividing the measurement light of the OCT optical system 100 and the anterior segment reflected light using the anterior segment illumination light source 402, and the scanning light scanned in the front imaging optical system 200 and having a wavelength band longer than λ=850 nm (preferably λ=900 nm). A cut-on wavelength is set in the first wavelength separation member 300 to be in a wavelength region in which the scanning light is unlikely to be visible for the subject eye, which is longer in wavelength than the wavelength band of the scanning light in the front imaging optical system 200, and which is shorter in wavelength than the wavelength band of the measurement light in the OCT optical system 100 and the wavelength band of the anterior segment reflected light using the anterior segment illumination light source 402 (the wavelength band of λ=930 nm to 950 nm inclusive).

Further, the present invention is not limited to the above configuration. The first wavelength separation member 300 may transmit the measurement light used in the OCT optical system 100, and may reflect the scanning light used in the front imaging optical system 200 and the anterior segment reflected light using the anterior segment illumination light source 402 (refer to FIG. 2). In this case, the first wavelength separation member 300 may have, for example, a wavelength selection characteristic for dividing the measurement light of the OCT optical system 100, and the anterior segment reflected light using the anterior segment illumination light source 402 and the scanning light scanned in the front imaging optical system 200 and having a wavelength band longer than λ=850 nm (preferably λ=900 nm). A cut-on wavelength is set in the first wavelength separation member 300 to be in a wavelength region in which the scanning light is unlikely to be visible for the subject eye, which is longer in wavelength than the wavelength band of the scanning light in the front imaging optical system 200 and the wavelength band of the anterior segment reflected light using the anterior segment illumination light source 402 (the wavelength band of λ=930 nm to 950 nm inclusive), and which is shorter in wavelength than the wavelength band of the measurement light of the OCT optical system 100.

<Second Wavelength Separation Member as Wavelength Selective Splitter>

In the fundus imaging apparatus, in a case where the anterior segment observation optical system 400 is provided, a second wavelength separation member 350, which performs wavelength separation on the optical path of one of the OCT optical system 100 and the front imaging optical system 200 divided by the first wavelength separation member in order to divide the optical path of the anterior segment observation optical system 400, may be provided as a wavelength selective splitter. In this case, for example, the second wavelength separation member 350 may perform wavelength separation in order to divide the optical path of the OCT optical system 100 and the optical path of the anterior segment observation optical system 400 (refer to FIG. 1). Alternatively, the second wavelength separation member 350 may perform wavelength separation in order to divide the optical path of the front imaging optical system 200 and the optical path of the anterior segment observation optical system 400.

For example, the second wavelength separation member 300 may have a wavelength characteristic in which the light used for one of the OCT optical system 100 and the front imaging optical system 200 divided by the first wavelength separation member is transmitted and the anterior segment reflected light using the anterior segment illumination light source 402 is reflected. For example, the second wavelength separation member 300 may have a wavelength characteristic in which the light used for one of the OCT optical system 100 and the front imaging optical system 200 divided by the first wavelength separation member is reflected and the anterior segment reflected light using the anterior segment illumination light source 402 is transmitted.

In a case where the optical path of the anterior segment observation optical system 400 is divided with respect to the OCT optical system 100 (refer to FIG. 1), the second wavelength separation member 350 may have, for example, a wavelength selection characteristic for dividing the measurement light in the OCT optical system 100 and the light in the wavelength band of the anterior segment reflected light using the anterior segment illumination light source 402, where the cut-on wavelength is set to be in a wavelength band longer than the wavelength band (wavelength band of λ=930 nm to 950 nm inclusive) of the anterior segment reflected light using the anterior segment illumination light source 402 and shorter than the wavelength band of the measurement light in the OCT optical system 100.

In a case where the optical path of the anterior segment observation optical system 400 is divided with respect to the front imaging optical system 200 (refer to FIG. 2), the second wavelength separation member 350 may have, for example, a wavelength selection characteristic for dividing the light in the wavelength band of the anterior segment reflected light using the anterior segment illumination light source 402 and the scanning light in the front imaging optical system 200, where the cut-on wavelength is set to be in a wavelength band shorter than the wavelength band (wavelength band of λ=930 nm to 950 nm inclusive) of the anterior segment reflected light using the anterior segment illumination light source 402 and longer than the wavelength band of the scanning light in the front imaging optical system 200.

<Wide-Angle Support>

As the light source 202 of the front imaging optical system 200, a light source that emits light having a central wavelength in a wavelength band longer than λ=900 nm and shorter than the measurement light (for example, λ=990 nm to 1100 nm inclusive) of the OCT optical system 100 may be used. In this case, it is necessary to set the cut-on wavelengths of the first wavelength separation member 300 and the second wavelength separation member 350 so as to be capable of separating the scanning light of the front imaging optical system 200, the anterior segment reflected light, and the measurement light of the OCT optical system 100 between 900 nm and 1000 nm.

In recent years, in the OCT optical system 100 and the front imaging optical system 200, the imaging angle of view (scanning angle of view) has been increased. In a case where the imaging angle of view is increased, the incident angle range of the OCT optical system 100 (or the front imaging optical system 200) with respect to the first wavelength separation member 300 and the second wavelength separation member 350 is increased. In this case, in a case where the incident angle with respect to the wavelength separation member is large, the light used in the OCT optical system 100 (or the front imaging optical system 200) has transmittance/reflectance characteristics sliding toward the short wavelength side when the optical path is divided by the first wavelength separation member 300 and the second wavelength separation member 350. That is, the cut-on wavelength for separating the measurement light of the OCT optical system 100 and the anterior segment reflected light, or the cut-on wavelength for separating the scanning light of the SLO optical system 200 and the anterior segment reflected light shifts to the short wavelength side. Further, as the angle of incidence on the wavelength separation member is smaller, the transmittance/reflectance characteristics shift to the longer wavelength side.

In this case, in consideration of sliding the cut-on wavelength to the short wavelength side and the long wavelength side, by using a light source that emits light having a central wavelength in the wavelength band longer than λ=900 nm and shorter than λ=910 nm (short wavelength region in the invisible region) as the light source 202 of the front imaging optical system 200, even in a case where the cut-on wavelength with the anterior segment observation optical system 400 shifts to the short wavelength side, it is easy to suppress the light amount loss of the scanning light of the front imaging optical system 200. Thereby, for example, a wide-angle front image of the fundus can be reliably obtained, and unevenness in the amount of light in the anterior segment image generated based on the anterior segment reflected light is reduced.

Further, in the anterior segment observation optical system 400, the cut-on wavelength with the OCT optical system 100 may shift to the short wavelength side by using the reflected light including at least the wavelength band of λ=930 nm to 950 nm inclusive as the reflected light of the anterior segment of the subject eye. Even in this case, it is easy to suppress the light amount loss of the measurement light of the OCT optical system 100. Thereby, for example, OCT data having a high resolution and a wide angle of view can be obtained by using a wide band measurement light, and unevenness in the amount of light in the anterior segment image generated based on the anterior segment reflected light is reduced.

In this case, the first wavelength separation member 300 and the second wavelength separation member 350 may have a wavelength characteristic which are set such that the reflected light including at least the wavelength band of λ=930 nm to 950 nm inclusive is guided to the imaging element 404 as the reflected light of the anterior segment of the subject eye.

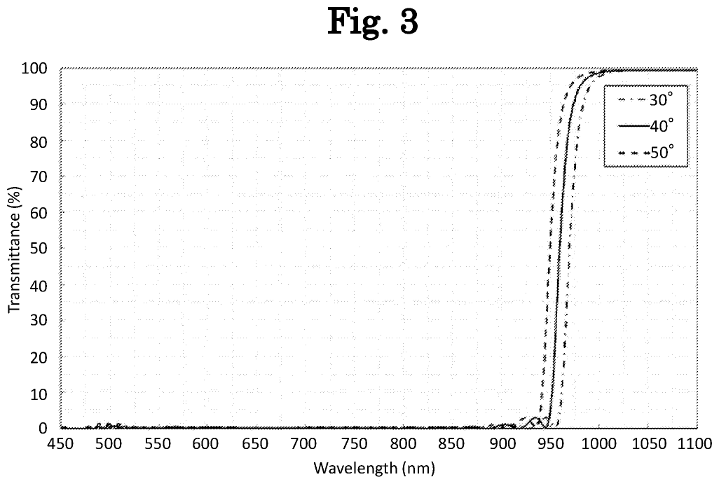

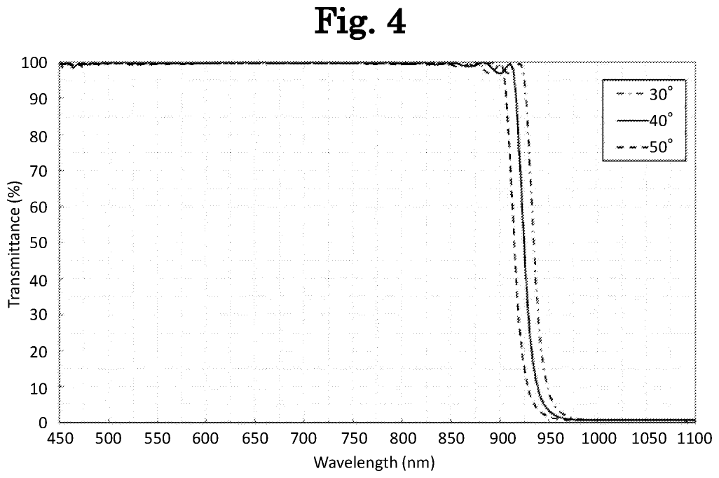

In the case of the optical arrangement of FIG. 1, for example, in the second wavelength separation member 350, the wavelength band longer than λ=950 nm (for example, λ=960 nm is set as the cut-on wavelength) which is the wavelength band shorter than the wavelength band of the measurement light of the OCT optical system 100 is set as the cut-on wavelength (refer to FIG. 3). Thereby, it is possible to increase the angle of the OCT optical system 100 while providing the anterior segment observation optical system 400. Further, in order to compensate for the light amount loss of the light used in the front imaging optical system 200 due to the optical path division, in the first wavelength separation member 300, the wavelength band shorter than λ=930 nm (for example, λ=925 nm is set as the cut-on wavelength), which is the wavelength band longer than the wavelength band of the scanning light of the front imaging optical system 200, is set as the cut-on wavelength (refer to FIG. 4). Thereby, it is possible to increase the angle of the front imaging optical system 200 (for example, it is possible to secure an imaging angle of 50 degrees or more) while providing the anterior segment observation optical system 400.

Further, in the case of the optical arrangement of FIG. 2, for example, in the first wavelength separation member 300, a wavelength band longer than λ=950 nm and shorter than the wavelength band of the measurement light of the OCT optical system 100 is set as the cut-on wavelength. Thereby, it is possible to increase the angle of the OCT optical system 100 while providing the anterior segment observation optical system 400. Further, in the second wavelength separation member 350, the cut-on wavelength is set in a wavelength band shorter than λ=930 nm and longer than the wavelength band of the scanning light of the front imaging optical system 200. Thereby, it is possible to increase the angle of the front imaging optical system 200 while providing the anterior segment observation optical system 400.

<Addition of Optical System Using Visible Light>

In the fundus imaging apparatus, the front imaging optical system 200 uses light in a wavelength band in which the scanning light is unlikely to be visible for the subject eye. Therefore, a wide wavelength region of visible light can be secured).

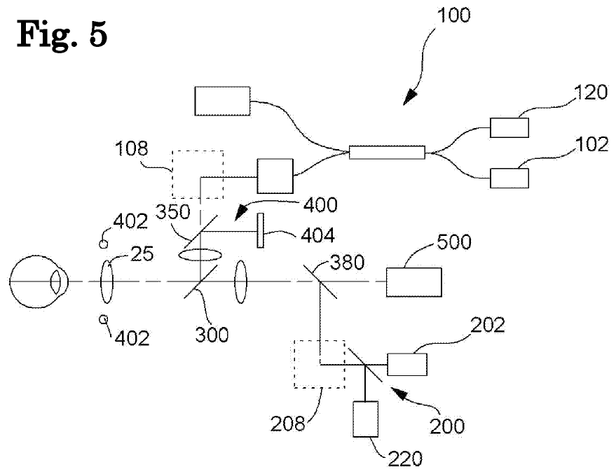

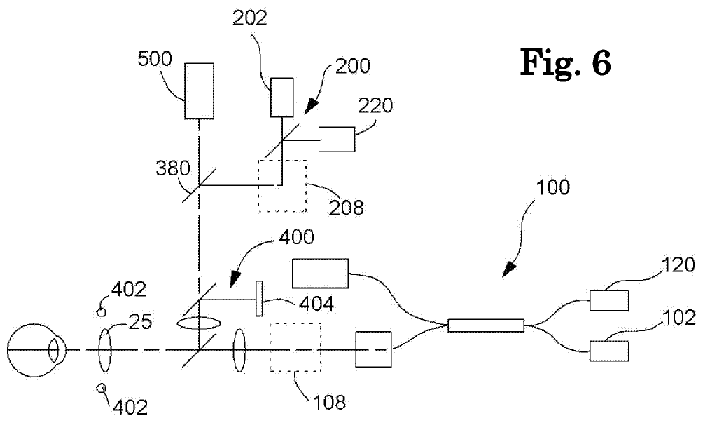

Therefore, as the fundus imaging apparatus according to the present example, for example, a visible optical system 500 that captures an image of the fundus or measures the fundus with visible light may be additionally provided (refer to, for example, FIGS. 5 and 6). In this case, the visible optical system 500 may include a visible light source that emits visible light having a central wavelength on the shorter wavelength side than the light used in the front imaging optical system 200. In this case, the visible optical system 500 may be used, for example, for fundus imaging or fundus stimulation. Further, as the visible optical system 500, for example, a visual fixation optical system that presents a visual fixation target (including a visual fixation lamp) for fixing a view of the subject eye may be provided.

The visible optical system 500 may be, for example, an imaging optical system for imaging the fundus with visible light. The imaging optical system may be, for example, a fundus camera type optical system or an SLO type optical system. In the fundus imaging apparatus including such a visible optical system, the front imaging optical system 200 is used, for example, for setting the imaging position and imaging timing of the front image of the fundus (fundus observation image) captured by the front imaging optical system 200. In this case, for example, the scanning light of the front imaging optical system 200 is in a wavelength region (preferably an invisible region) in which the scanning light is unlikely to be visible. Therefore, the scanning light makes the subject eye unlikely to track, and it is possible to obtain a favorable visible fundus image. More specifically, the fundus can be observed using the clear front image obtained by the scanning type front imaging optical system 200. Therefore, it is possible to accurately confirm the imaging timing, the imaging position, and the like. In addition, the scanning light of the scanning type front imaging optical system 200 is unlikely to be visible, and the subject eye is unlikely to track the scanning light. Therefore, it is possible to reliably perform the fundus imaging with visible light.

The visible optical system 500 may be a measurement optical system for projecting a stimulus point onto the fundus with visible light to perform visual field measurement. In the fundus imaging apparatus including such a visible optical system, the front imaging optical system can be used in, for example, setting of the stimulation position on the front image of the fundus captured by the front imaging optical system, position correction (tracking) of a stimulus point based on the front image of the fundus captured by the front imaging optical system, and the like. In this case, the scanning light of the front imaging optical system is in a wavelength region (preferably an invisible region) in which the scanning light is unlikely to be visible. Therefore, the scanning light makes the subject eye unlikely to track, and it is possible to obtain favorable visual field data. More specifically, the measurement point of the visual field can be set using the clear front image obtained by the scanning type front imaging optical system 200. Therefore, the measurement point can be set accurately, the scanning light is unlikely to be visible, and the subject eye is unlikely to track the scanning light. As a result, it is possible to reliably perform visual field measurement.

In a case where the visible optical system 500 is provided in the fundus imaging apparatus, the visible optical system 500 is provided between the optical scanner 208 provided in the front imaging optical system 200 and the subject eye. A third wavelength separation member 380 that performs wavelength separation for dividing the optical path of the OCT optical system 100 and the optical path of the front imaging optical system 200 and the visible optical system 500 may be provided therein as a wavelength selective splitter.

FIG. 5 shows an example in which the visible optical system 500 is provided in the fundus imaging apparatus having the optical arrangement of FIG. 1. The third wavelength separation member 380 may be disposed upstream (on the light source side) of the first wavelength separation member 300 to divide the optical path of the visible optical system 500 and the optical path of the front imaging optical system 200, in the transmission direction of the first wavelength separation member 300.

FIG. 6 shows an example in which the visible optical system 500 is provided in the fundus imaging apparatus having the optical arrangement of FIG. 2. The third wavelength separation member 380 may be disposed upstream (on the light source side) of the second wavelength separation member 350 to divide the optical path of the visible optical system 500 and the optical path of the front imaging optical system 200, in the transmission direction of the second wavelength separation member 350.

According to the optical arrangement as shown in FIGS. 5 and 6, the wavelength separation members 300, 350, and 380 can be supported without forming a band bus. Therefore, efficient manufacturing is possible.

### Example of Transformation

In the above examples, in the fundus imaging apparatus including the OCT optical system 100 and the front imaging optical system 200, a wavelength region (preferably an invisible region), in which the scanning light of the front imaging optical system 200 is unlikely to be visible for the subject eye, is used. But, the present disclosure is not limited to the examples.

For example, in a fundus imaging apparatus including the visible optical system 500 and the front imaging optical system 200, a wavelength region (preferably an invisible region), in which the scanning light of the front imaging optical system 200 is unlikely to be visible for the subject eye, may be used. Thereby, for example, the scanning light makes the subject eye unlikely to track. As a result, it is possible to reliably perform imaging or measurement with visible light.

### Example of Second Embodiment

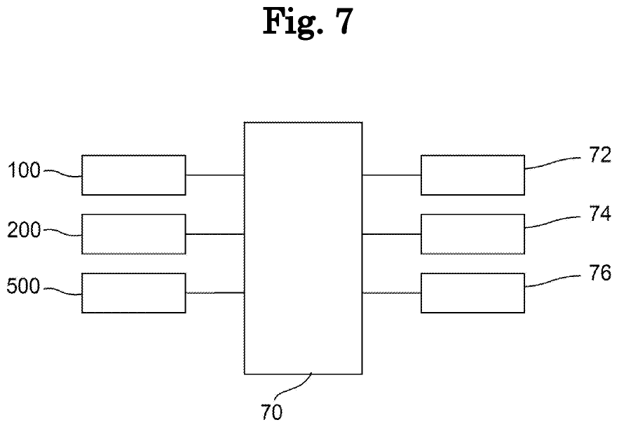

Hereinafter, examples according to the second embodiment will be described with reference to the drawings. As the optical system according to the present example, for example, the configuration shown in FIG. 5 or FIG. 6 can be used. FIG. 7 is a block diagram showing an example of the control system according to the present example. Unless otherwise specified, it is assumed that those having the same numerals as those of the examples according to the first embodiment have the same functions and configurations.

In the case of the fundus imaging apparatus according to the present example, as the visible optical system 500, a measurement optical system for projecting a stimulus point onto the fundus with visible light to perform visual field measurement is used. Hereinafter, the visible optical system 500 will be described as the measurement optical system 500. That is, the fundus imaging apparatus includes, for example, the measurement optical system 500, the respective optical systems including the OCT optical system 100, the front imaging optical system 200, and the anterior segment observation optical system which is not shown are connected to a controller 70. Further, the controller 70 is connected to a response reception unit 72, a display unit 74, a storage unit 76, and like that receive a response signal from the subject. The response reception unit 72 may be operated by the subject.

<Measurement Optical System 500>

The measurement optical system 500 includes a visual target projection optical system that projects a visual stimulation target within the visual field of the subject eye. The measurement optical system 500 includes, for example, a display and a light guide optical system that guides light from the display to the fundus. The controller 70 controls the measurement optical system 500 such that the measurement optical system 500 projects (presents) the visual stimulation target onto the subject eye, acquires the response signal from the response reception unit 72 at each stimulus point, and measures the visual field of the subject eye, based on the response result acquired the response signal at each stimulus point.

In this case, the controller 70 may control the measurement optical system 500 based on a preset visual field examination pattern. The visual field examination pattern may be composed of predetermined examination conditions. As examination conditions, for example, the visual field measurement range, the position of each stimulus point, the initial reference luminance at each stimulus point, the projection order of the stimulus points, and the like may be preset. The controller 70 measures the visual field in accordance with the set visual field examination pattern. The visual field examination pattern may be changed based on the response signal from the subject (for example, utilization of a time saving algorithm). As the visual field examination pattern, a single or a plurality of visual field examination patterns may be provided in advance as a template, or an arbitrary visual field examination pattern may be created before the start of the visual field measurement. The visual field examination pattern may be composed of examination conditions for the purpose of static visual field measurement or may be composed of examination conditions for the purpose of dynamic visual field measurement.

<OCT Optical System 100>

The controller 70 controls the OCT optical system 100, based on a preset imaging control pattern. As the imaging conditions constituting the imaging control pattern, the following are preset: for example, a scanning range (for example, scanning width on the fundus, scanning angle of view); a scanning position (for example, macula, papilla, and periphery of the fundus); a scanning pattern (for example, line scan consisting of one scanning line, multi-scan consisting of multiple scanning lines, circle scan, raster scan, and like); an imaging mode (for example, normal imaging, adding averaging imaging, wide-angle imaging, OCT angiography, OCT Doppler imaging, and like); and like. In this case, the imaging control pattern may be composed of a single imaging condition or a plurality of imaging conditions. The imaging control pattern composed of a plurality of imaging conditions, for example, may be a control pattern for performing a first imaging control step of acquiring the OCT data under the first imaging condition and a second imaging control step of acquiring the OCT data under the second imaging condition different from the first imaging condition. In this case, the imaging control pattern may be a control pattern for acquiring the OCT data under a large number of (at least three or more) imaging conditions.

In this case, the imaging control pattern may be set assuming that OCT imaging is performed during visual field measurement that requires a long time. For example, imaging control pattern may be a control pattern in which it takes a long time to perform the OCT imaging when performing the imaging control pattern. For example, the following is conceivable. OCT angiography is performed in a high density and a wide range, addition average imaging in which a large number of sheets are added is performed in a high density and a wide range, OCT panoramic imaging consisting of a plurality of raster scans is performed in a high density and a wide range, imaging using all scanning patterns is performed, and so on. In other words, it takes a long time to perform the OCT imaging due to the following causes: high-density scanning density; wide-ranging scanning range, imaging using a large number of scanning patterns; OCT angiography and OCT Doppler imaging that require multiple scans in a two-dimensional region; and like. However, by proceeding in parallel with the visual field examination that requires a long time, it is possible to substantially offset the time required for the OCT imaging (details will be described later). It should be noted that the collection of OCT data during the visual field measurement is particularly advantageous for substantially shortening the OCT imaging time having a long time. However, even in the OCT imaging performed in a relatively short time (for example, acquisition of one OCT data only), application of the present example is possible.

<Operation Explanation>

Hereinafter, in the fundus imaging apparatus having the above configuration, an operation in a case of collecting the OCT data during a visual field examination will be described. An example of measuring a static visual field will be given.

In the stage before starting the visual field examination and OCT imaging, the examiner presets the visual field examination pattern and the imaging control pattern. For example, a plurality of patterns are provided as initial settings for each of the visual field examination pattern and the imaging control pattern, and at least one of the plurality of patterns is selected. It is apparent that the pattern is not limited to the pattern in the initial setting, and the visual field examination conditions or the OCT imaging conditions may be set in detail.

In a case where the alignment of the apparatus with respect to the subject eye is completed, the controller 70 does not move the index and fixes the subject eye at a certain position. The visual field measurement is automatically or manually started based on a predetermined trigger signal, and the controller 70 controls to sequentially illuminate spot light toward a plurality of stimulus points on the fundus, based on a preset visual field examination pattern. Then, the response result is obtained for each stimulus point. For example, the controller 70 controls to fix and present the visual target at a specific point in the visual field of the subject eye, and controls to present the visual target at another point so as to calculate the brightness identification threshold value (visual sensitivity threshold) of the visual target over a wide range of the visual field in accordance with the visual field response.

In the present example, the controller 70 controls the OCT optical system 100 based on a preset imaging control pattern while each stimulus point is projected onto the fundus as described above, thereby collecting the OCT data of the fundus of the subject eye.

The collection of the OCT data is started automatically or manually based on a predetermined trigger signal, and the controller 70 collects the OCT data, based on a preset imaging control pattern. The OCT data collection timing may be the same as the start of the visual field examination, or may be another timing. In a case of collecting the OCT data, in addition to alignment with respect to the subject eye, focus adjustment, optical path length adjustment, polarization adjustment, and like are required (so-called optimization). Therefore, after these controls are completed, the OCT data collection may be started.

The collected OCT data is displayed on the display unit 74 in a case where the acquisition is completed. Further, the controller 70 causes the storage unit 76 to store the collected OCT data at any time.

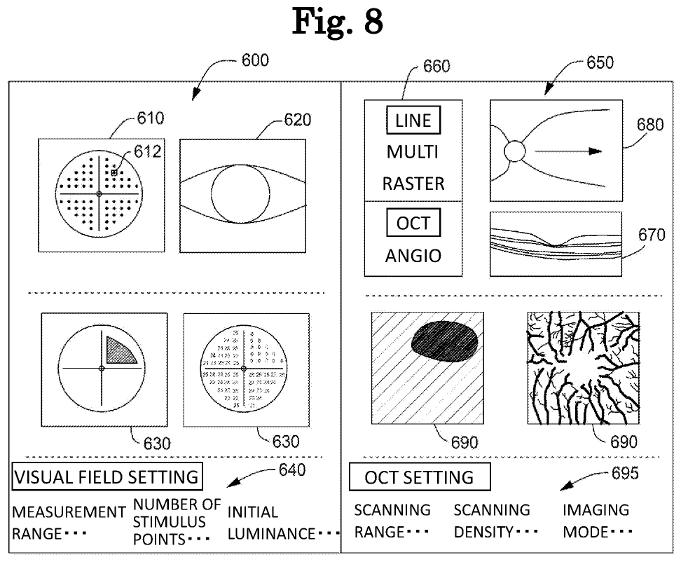

FIG. 8 is a diagram showing an example of a display screen of the apparatus according to the present example. For example, the controller 70 may display the visual field examination display region 600 (hereinafter, visual field display region 600) and the OCT examination display region 650 (hereinafter, OCT display region 650) on the same screen of the display unit 74.

In the visual field display region 600, for example, a visual field measurement chart 610 and a front image observation screen 620 are displayed. For example, a graphic showing the projection position of the stimulus point is displayed on the visual field measurement chart 610. In this case, at least the index 612 indicating the current position of the stimulus point is displayed during the visual field measurement. In the pre-examination stage, the visual field measurement chart 610 may display the position of each stimulus point corresponding to the preset visual field examination pattern.

For example, the front image of the anterior segment captured by the anterior segment observation optical system or the front image of the fundus captured by the front imaging optical system is displayed as a moving image in live view, on the front image observation screen 620, and the state of the subject eye during visual field measurement may be observable in real time. In a case where the front image of the fundus is displayed, the visual field measurement chart 610 may be superimposed on the front image of the fundus.

After the visual field measurement is started, the controller 70 displays an index corresponding to the position of the current stimulus point on the visual field measurement chart 610 while each stimulus point is projected onto the fundus. That is, the position of the index on the visual field measurement chart 610 is changed in accordance with the position change of the stimulus point. In this case, as the display form of the index, the stimulus points being projected may be highlighted in a state where each set stimulus point is displayed.

In the OCT display region 650, for example, an imaging condition display screen 660, an OCT image display screen 670, and a front image observation screen 680 are displayed. For example, the imaging conditions for acquiring the OCT data by the OCT optical system 100 are displayed on the imaging condition display screen 660. In this case, the current imaging conditions of the OCT data are displayed during the acquisition of the OCT data. In the stage before data acquisition, the imaging condition display screen 660 may display the imaging conditions corresponding to the preset imaging control patterns as described above. In this case, a plurality of imaging conditions may be displayed in a list.

On the OCT image display screen 670, for example, the OCT data acquired by the OCT optical system 100 may be displayed as a moving image in live view. Further, for example, the front image of the fundus acquired by the front imaging optical system 200 may be displayed as a moving image in live view, on the front image observation screen 680.

After the OCT imaging is started, the controller 70 displays the current imaging conditions, on the imaging condition display screen 660. In this case, the display of the imaging conditions may be updated in accordance with the change of the imaging conditions. In this case, as the display form of the imaging conditions, the imaging conditions during data acquisition may be highlighted while a list of a plurality of imaging conditions is displayed. Further, the controller 70 displays the OCT data and the front image of the fundus in real time on the OCT image display screen 670 and the front image observation screen 680.

According to the above display screen, for example, when the OCT data is acquired during visual field measurement, it is possible to simultaneously grasp both the status of visual field examination performed based on a preset visual field examination pattern and the status of the OCT examination performed based on a preset imaging control pattern.

Here, in a case where a predetermined release signal is automatically or manually input, the controller 70 acquires the OCT data (or the front image of the fundus) as a still image and causes the storage unit 76 to store the OCT data, and displays the acquired still image on the display unit 74. In this case, a switch for executing re-imaging may be disposed in order to deal with the case where imaging for the acquired OCT data (or the front image of the fundus) fails.

In the visual field display region 600, for example, an analysis result 630 (for example, a visual field analysis map) based on the visual field examination result may be displayed. As the visual field analysis map, for example, a map showing the visual sensitivity at each stimulus point in two dimensions may be displayed. In this case, the analysis result based on the already acquired visual field examination result may be displayed even during the visual field measurement.

In the OCT display region 650, for example, an analysis result 690 based on the OCT data (for example, an OCT analysis map, an OCT analysis chart, an OCT front image) may be displayed. As the OCT analysis map, for example, a thickness map two-dimensionally showing the thickness of the fundus at each position and a blood vessel density map two-dimensionally showing the blood vessel density at each position may be displayed. The analysis chart may be, for example, a chart in which the analysis result is divided for each analysis region and the integrated value (for example, average value and maximum value) for each analysis region is displayed. Further, the OCT front image may be, for example, an en-face OCT image that two-dimensionally shows at least a part of the luminance in the depth direction of the fundus, or an en-face OCT angio image based on OCT motion contrast data. In this case, the analysis result based on the already acquired OCT data may be displayed even during the visual field measurement and the OCT imaging.

In the visual field display region 600, for example, a field of visual field setting screen 640 for the examiner to set a visual field measurement pattern may be displayed, and the controller 70 may receive a setting instruction from the examiner through an operation on the setting screen. In this case, the setting screen may be used for not only setting the measurement pattern before starting the visual field examination but also, for example, changing the measurement pattern during measurement, or performing additional examination.

The OCT setting screen 695 for the examiner to set the imaging control pattern may be displayed in the OCT display region 650, and the controller 70 may receive a setting instruction from the examiner through an operation on the setting screen. In this case, the setting screen may be used for not only setting an imaging control pattern before starting acquisition of the OCT data but also, for example, changing an imaging pattern, or performing additional imaging.

According to the above, the visual field setting screen is displayed during the visual field measurement. Thereby, for example, by using a moving image or the analysis result based on the OCT data obtained during visual field examination, it is possible to perform additional visual field examination and change examination pattern. In this case, in the additional examination, in a case where the additional examination is executed after the examination using the visual field examination pattern which is set before the measurement is completed and there is an instruction to change the pattern, the examination pattern may be changed during the examination.

Further, the OCT setting screen is displayed during the visual field measurement. Thereby, for example, by using the analysis result of the visual field examination, it is possible to perform additional OCT imaging and change the imaging control pattern. In this case, in the additional OCT imaging, in a case where the additional imaging is executed after the imaging according to the imaging control pattern which is set before the measurement is completed and there is a pattern change instruction, the imaging control pattern may be changed during imaging.

In a case where the series of visual field examinations and OCT imaging are completed as described above, the controller 70 displays the visual field examination results and the OCT data acquired so far on the display unit 74. In this case, the controller 70 may display various OCT data obtained during the visual field measurement side by side on the display unit 74, or may simultaneously display the analysis result of the visual field or the OCT data described above. Based on these results, the examiner is able to evaluate the presence or absence of fundus disease, the progress, and the like of the subject eye from both the functional and structural aspects of the fundus.

According to the above configuration, the OCT data can be collected by utilizing the time required for visual field examination that takes a long term to perform. Therefore, it is possible to acquire a large number of various OCT data regardless of the time required for OCT imaging, and it is possible to perform imaging in an imaging mode that requires relatively long imaging time, with a margin. Thereby, clinically useful OCT data and a large number of OCT data necessary for disease research can be obtained without burdening the examiner and the subject as compared with the conventional case.

<Automatic Setting of Stimulus Point According to OCT Data Collection Result>

In a case where the position of the stimulus point is set using the OCT data, for example, the controller 70 may process the OCT data collected during the visual field examination and acquire the position information regarding the abnormal part of the fundus so as to set the position of the stimulus point based on the acquisition position information.

In this case, the controller 70 may analyze the OCT data using artificial intelligence and specify the position of the abnormal part on the fundus. Further, the controller 70 may analyze the analysis result obtained by processing the OCT data by using artificial intelligence and specify the position of the abnormal part on the fundus. In specifying the abnormal part, an attention map showing the probability of the disease two-dimensionally may be generated based on the OCT data, and the position where the probability of the disease is high in the attention map may be specified as the abnormal part. The attention map may be displayed on the display unit 74 and used for setting the position of the stimulus point and the luminance. It is apparent that it is not always necessary to use artificial intelligence in specifying the abnormal part. For example, the abnormal part may be specified by using the thickness information of the fundus, the blood vessel density information, and the like.

In a case where the abnormal part is specified, for example, the controller 70 may set the position of the stimulus point at a predetermined interval in the region including the abnormal part in order to obtain the visual field measurement result in the abnormal part in detail. In this case, the controller 70 may change the preset visual field examination pattern before start of the visual field examination so as to increase the number of stimulus points, and may perform the visual field measurement on the abnormal part, additionally.

By using the OCT data collected during the visual field examination as described above, it is possible to improve the efficiency of both the OCT examination and the visual field examination as a result. It should be noted that details on setting the stimulus point based on the OCT data, refer to, for example, JP-A-2012-100713 applied by the applicant.

In the above description, the position of the stimulus point is set using the OCT data collected during the visual field examination, but the present invention is not limited to this. For example, the OCT data may be acquired before the visual field examination, and the visual field examination pattern may be set based on the acquired OCT data. Then, the controller 70 may further collect the OCT data while each stimulus point is projected onto the fundus using a visual field examination pattern based on the OCT data.

<Setting of OCT Imaging Condition Based on Visual Field Measurement Result>

In a case where the acquisition position of the OCT data is set using the visual field measurement result, for example, the controller 70 may process the measurement result data acquired by the visual field examination and acquire the position information regarding the abnormal part of the fundus so as to set the acquisition position of the OCT data based on the acquired position information.

In this case, the controller 70 may acquire position information regarding the abnormal part of the fundus by performing determination processing on the acquired measurement result data using a predetermined determination criterion, and may set the acquisition position of the OCT data such that the abnormal part is included in the imaging region. It should be noted that the specific method refer to, for example, JP-A-2012-100714. The controller 70 may analyze the visual field examination result using artificial intelligence and specify the position of the abnormal part on the fundus. Further, the controller 70 may analyze the analysis result (for example, the visual field analysis map), which is obtained by processing the visual field examination result, through artificial intelligence, and may specify the position of the abnormal part on the fundus. In specifying the abnormal part, an attention map showing the probability of the disease two-dimensionally may be generated based on the visual field examination result, and the position where the probability of the disease is high in the attention map may be specified as the abnormal part. The attention map may be displayed on the display unit 74 and may be used for setting the acquisition position of the OCT.

In a case where the abnormal part is specified, for example, the controller 70 may acquire high-density OCT data in the region including the abnormal part in order to obtain the OCT data in the abnormal part in detail, or may acquire the OCT data (for example, OCT motion contrast data) in a specific imaging mode in the region including the abnormal part. In this case, the controller 70 may change the imaging conditions constituting the preset imaging control pattern before the start of the OCT imaging, or may additionally perform the OCT imaging on the abnormal part.

With such a configuration mentioned above, when acquiring the OCT data during visual field measurement, by using the visual field examination result already obtained during visual field measurement, as a result, it is possible to improve efficiency of both the OCT imaging and the visual field examination. In addition, since detailed OCT data corresponding to the visual field examination is acquired, it is possible to evaluate the abnormal part in terms of function and structure in more detail.

<OCT Measurement Before and After Light Stimulation>

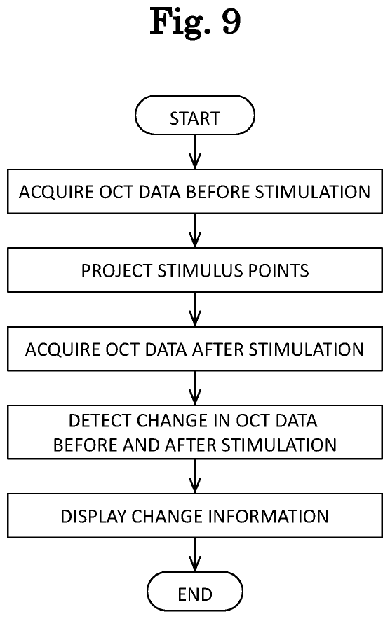

FIG. 9 is a flowchart showing an example of detecting a change in OCT data before and after light stimulation. The controller 70 may detect a change in OCT data before and after light stimulation using the stimulus point by controlling the OCT optical system 100 while each stimulus point is projected onto the fundus. Since the visual field examination pattern is preset, the position and stimulation timing of each stimulus point are known. The controller 70 controls the optical scanner 108 of the OCT optical system 100, and acquires the OCT data before light stimulation in the fundus region including the first stimulus point before the first stimulus point is projected onto the fundus. Next, the controller 70 controls the measurement optical system 500 and projects the first stimulus point onto the fundus. After the first stimulus point is projected onto the fundus, the controller 70 controls the optical scanner 108 of the OCT optical system 100, and acquires the OCT data after light stimulation in the fundus region including the first stimulus point. In this case, the OCT data including stimulus points may be acquired continuously in time, and for example, the OCT data during light stimulation may be acquired. In a case where the measurement is performed at the stimulus point at the same position with different luminance, the OCT data before and after the stimulation may be acquired again, or may be captured together with the change of the OCT data.

Next, the visual field is measured at the first stimulus point, and the OCT data before and after the stimulation at the first stimulus point is acquired. Thereafter, when the position of the stimulus point is changed from the first stimulus point to the second stimulus point, the controller 70 acquires the OCT data before light stimulation in the fundus region including the second stimulus point, before the second stimulus point is projected onto the fundus. Next, the controller 70 projects the second stimulus point onto the fundus. After the second stimulus point is projected onto the fundus, the controller 70 acquires the OCT data after light stimulation in the fundus region including the second stimulus point. Thereby, the visual field is measured at the second stimulus point, and the OCT data before and after the stimulation at the second stimulus point is acquired.

As described above, the controller 70 acquires the OCT data before and after the light stimulation at each stimulus point while each stimulus point is projected onto the fundus. Next, the controller 70 may obtain the change in luminance of the OCT data before and after the light stimulation from each pixel for each stimulus point. The change in luminance can be obtained by obtaining a difference, a ratio, or the like. By detecting the change in luminance of the OCT data in such a manner, the intrinsic signal of the fundus for each stimulus point is extracted. The controller 70 displays the change in luminance information at each stimulus point on the display unit. In this case, the OCT data may be displayed corresponding to each pixel, or the integrated value of the change in luminance (for example, the average value, the maximum value, and like) may be displayed.

Since the intrinsic signal of the fundus as described above can be considered as a retinal reaction caused by light stimulation, the retinal sensitivity at each stimulus point can be objectively confirmed by capturing the intrinsic signal at each stimulus point. For example, in a case where the sensitivity of the retina is high, the intrinsic signal is considered to be strong, and in a case where the retina sensitivity is low, the intrinsic signal is considered to be weak. Thereby, it is possible to objectively measure the visual field at each stimulus point. In this case, by acquiring the subjective response result from the examiner, the relationship between the subjective response result and the intrinsic signal can be evaluated. Further, the objective visual field measurement based on the intrinsic signal may be performed without acquiring the subjective response result.

In the above description, the change in luminance of the OCT data before and after the light stimulation is detected, but the present invention is not limited to this. For example, in a case where detecting a change in the OCT data, the OCT data before the light stimulation and the OCT data after the light stimulation may be analyzed to detect the change in the analysis result. In this case, a change in the thickness of the fundus may be detected. According to this, the structural change of the fundus before and after the light stimulation can be evaluated.

The OCT data acquired before and after the light stimulation may be B scan OCT data or three-dimensional OCT data. In a case where the OCT data is the B scan OCT data, it is possible to acquire the data in a short time. In a case where the OCT data is the three-dimensional OCT data, a change in OCT data including the periphery of the stimulus point can be obtained in two dimensions. In this case, by obtaining the OCT data in a certain range including the center and the periphery of the stimulus point, it is possible to evaluate the influence on the surrounding tissues in addition to the change of the photoreceptor cells by the light stimulation.

In the above, the exemplary case where the OCT data is a normal OCT image showing the reflection intensity (scattering intensity) of the measurement light on the fundus is illustrated. However, the OCT data may be OCT motion contrast data, and may be the OCT Doppler data. In this case, the OCT motion contrast data is shown as, for example, data in which the amount of change in amplitude, phase, and like between the OCT data pieces that differ with time is imaged as a luminance value. The OCT Doppler data is, for example, data based on the Doppler shift detected by comparing the OCT data different in time, and is used for acquiring the blood flow velocity of a blood vessel or the like. That is, the controller 70 may detect the change in the OCT motion contrast data before and after the light stimulation (for example, the change in luminance between the OCT motion contrast data pieces), or may detect the change in the OCT Doppler data before and after the light stimulation (for example, the change between the OCT Doppler data pieces).

In this case, the controller 70 is able to acquire the OCT motion contrast data (or OCT Doppler data) before the light stimulation by acquiring a plurality of OCT data pieces that are temporally different at the same position before the light stimulation. Further, the controller 70 is able to acquire the OCT motion contrast data (or OCT Doppler data) after the light stimulation by acquiring a plurality of OCT data pieces that are temporally different at the same position after the light stimulation. The method of acquiring OCT motion contrast data and OCT Doppler data, refer to JP-A-2016-106651 and JP-A-2018-121888 applied by the applicant. In this case, the OCT motion contrast data may be acquired in advance in order to obtain the three-dimensional structure of the fundus blood vessel which is the basis of the OCT Doppler data.

For example, the controller 70 may obtain the change in luminance of the OCT motion contrast data before and after the light stimulation. Thereby, the intrinsic signal based on the OCT motion contrast data may be confirmed. Further, the change in the blood vessel structure imaged by the OCT motion contrast data (for example, the change in blood vessel diameter, and the change in the number of capillaries) may be confirmed. In this case, by obtaining the difference in luminance, the increase/disappearance of the imaged blood vessels can be detected. As a result, the change in the blood vessel diameter and the change in number of capillaries can be captured.

In a case of acquiring OCT motion contrast data before and after stimulation, the controller 70 may acquire three or more OCT data pieces that are temporally different with respect to the same position before and after stimulation, and may create OCT motion contrast data for each different time interval between the OCT data pieces. In this case, since the time interval differs depending on the group, blood vessels having different blood flow velocities can be detected as motion contrast. In this case, the controller 70 may detect changes between the OCT motion data pieces before and after stimulation (for example, the change in luminance and the change in blood vessel structure) by dividing the time interval between the underlying the OCT data pieces. The method of obtaining OCT motion contrast data having different time intervals refer to International Publication No. WO2017/119437 applied by the applicant.

The controller 70 may analyze the OCT motion contrast data before the light stimulation and the OCT motion contrast data after the light stimulation, respectively, and detect a change in the analysis result. In this case, the change in blood vessel density may be detected. According to this, the structural change of the fundus blood vessel before and after the light stimulation can be evaluated.

The controller 70 may detect the change in blood flow velocity before and after the stimulation by detecting the change in the OCT Doppler data before and after the light stimulation (for example, the change between the OCT Doppler data pieces). Thereby, the intrinsic signal based on the OCT Doppler data may be confirmed.

In the above example, the OCT optical system may be an OCT optical system (AO-OCT optical system) including a wavefront compensation optical system capable of compensating for higher-order aberrations of the subject eye. In this case, evaluation using the OCT data can be performed in more detail at the cellular level. In this case, the wavefront compensation optical system may also be provided in the front imaging optical system.

In the above example, the fundus imaging apparatus including the visual field measurement optical system and the OCT optical system has been described. However, the present embodiment may also be applied to an anterior segment imaging device including an eye refractive power measurement optical system for measuring the accommodation ability of the subject eye and an OCT optical system for acquiring the OCT data of a crystalline lens of the anterior segment of the subject eye. In this case, in addition to the above-mentioned fundus imaging apparatus, there may be provided an eye refractive power measurement optical system and a configuration for acquiring the OCT data of the anterior crystalline lens (for example, a mechanism for switching the fundus and the anterior segment by performing lens insertion/removal or attachment). In this case, it is possible to detect the result of the accommodation ability measurement and the change in the OCT data during the accommodation.

What is claimed is:

1. A fundus imaging apparatus comprising:
an OCT optical system configured to be used for acquiring OCT data based on measurement light irradiated on a fundus of a subject eye and reference light; and
a front imaging optical system configured to be used for capturing a front image of the fundus of the subject eye by scanning light on the fundus,
wherein the scanning light scanned on the fundus of the subject eye in the front imaging optical system has a wavelength, which is longer than λ=850 nm, in a wavelength region in which a scanning light is unlikely to be visible for the subject eye.

2. The fundus imaging apparatus according to claim 1,
wherein the scanning light scanned on the fundus of the subject eye in the front imaging optical system has a wavelength, which is longer than λ=900 nm, in an invisible region for the subject eye.

3. The fundus imaging apparatus according to claim 1,
wherein a light source used in the OCT optical system is a light source that emits the measurement light having a central wavelength between λ=1000 nm to 1100 nm inclusive.

4. The fundus imaging apparatus according to claim 2, further comprising:
an anterior segment observation optical system that guides light, which is illuminated by an anterior segment illumination light source and reflected on an anterior segment of the subject eye, to an imaging element;
a first wavelength separation member that performs wavelength separation to divide an optical path of the measurement light in the OCT optical system and an optical path of the scanning light in the front imaging optical system and to divide an optical path of the anterior segment observation optical system with respect to one of optical paths of the OCT optical system and the front imaging optical system; and
a second wavelength separation member that performs wavelength separation to divide the optical path of the anterior segment observation optical system with respect to one of the optical paths of the OCT optical system and the front imaging optical system divided by the first wavelength separation member.

5. The fundus imaging apparatus according to claim 4,
wherein the first wavelength separation member and the second wavelength separation member are set to have wavelength characteristics in which reflected light including at least a wavelength band of λ=930 nm to 950 nm inclusive is guided to the imaging element as the light reflected on the anterior segment of the subject eye.

6. The fundus imaging apparatus according to claim 1, further comprising:
a visible optical system that includes a visible light source emitting visible light having a central wavelength on a shorter wavelength side than light used for the front imaging optical system; and
a third wavelength separation member that is provided between the subject eye and an optical scanner provided in the front imaging optical system and performs wavelength separation to divide optical paths of the front imaging optical system and the visible optical system and an optical path of the OCT optical system.

7. A fundus imaging apparatus comprising:
a measurement optical system that projects a stimulus point onto a fundus of a subject eye with visible light to measure a visual field; and
a front imaging optical system configured to be used for capturing a front image of the fundus of the subject eye by scanning light on the fundus,
wherein the scanning light scanned on the fundus of the subject eye in the front imaging optical system has a wavelength, which is longer than λ=850 nm, in a wavelength region in which a scanning light is unlikely to be visible for the subject eye.

8. The fundus imaging apparatus of claim 7, further comprising:
an OCT optical system configured to be used for acquiring OCT data based on measurement light irradiated on the fundus of the subject eye and reference light; and
a controller configured to control the OCT optical system to collect the OCT data of the fundus of the subject eye while each of a plurality of stimulus points is projected onto the fundus of the subject eye by controlling the measurement optical system based on a visual field examination pattern in which the plurality of stimulus points are preset.

9. The fundus imaging apparatus according to claim 8,
wherein a light source used in the OCT optical system is a light source that emits measurement light having a central wavelength in a wavelength region which is an invisible region for the subject eye.

10. The fundus imaging apparatus according to claim 8,
wherein the controller sets an acquisition position of OCT data acquired in the OCT optical system, based on a visual field measurement result measured while each stimulus point is projected onto the fundus of the subject eye.

11. The fundus imaging apparatus according to claim 8,
wherein the controller sets a position of each stimulus point in the measurement optical system, based on a collected result of OCT data collected while each stimulus point is projected onto the fundus of the subject eye.

12. The fundus imaging apparatus according to claim 8,
wherein the controller performs light stimulation on the fundus of the subject eye by controlling the measurement optical system or a light stimulation unit different from the measurement optical system, and detects a temporal change of OCT data before and after the light stimulation.

13. The fundus imaging apparatus according to claim 8,
wherein the controller changes an acquisition position of OCT data in accordance with a position of each stimulus point such that the OCT data at each stimulus point is acquired.

14. A fundus imaging apparatus comprising:
a visible imaging optical system configured to be used for imaging a fundus of a subject eye with visible light; and
a front imaging optical system configured to be used for capturing a front image of the fundus of the subject eye by scanning light on the fundus,
wherein the scanning light scanned on the fundus of the subject eye in the front imaging optical system has a wavelength, which is longer than λ=850 nm, in a wavelength region in which a scanning light is unlikely to be visible for the subject eye.

15. The fundus imaging apparatus according to claim 14,
wherein the scanning light scanned on the fundus of the subject eye in the front imaging optical system has a wavelength, which is longer than λ=900 nm, in an invisible region for the subject eye.
